(19) United States
(12) Patent Application Publication
(10) Pub. No.: US 2011/0301202 A1
(43) Pub. Date: Dec. 8, 2011

(54) SPLA2 INHIBITOR CONJUGATE COMPOUNDS AND METHODS OF USE

(76) Inventors: Colin Hislop, Menlo Park, CA (US); Joaquim Trias, Millbrae, CA (US); Debra Odink, Oakland, CA (US); Bernadine Fraser, Sunnyvale, CA (US); Scott Chadwick, Reno, NV (US)

(21) Appl. No.: 13/152,689

(22) Filed: Jun. 3, 2011

Related U.S. Application Data
(60) Provisional application No. 61/351,817, filed on Jun. 4, 2010.

(51) Int. Cl. A61K 31/4439; A61P 3/06; C07D 401/12

(52) U.S. Cl. 514/339; 546/278.1

(57) ABSTRACT

The present application provides a novel set of compounds comprising a sPLA₂ inhibitor conjugated to a niacin drug, as well as kits containing these compounds and methods of using the compounds to alter lipid levels and treat various cardiovascular diseases.

### RELATED APPLICATIONS

The present application claims priority to U.S. Provisional Patent Application No. 61/351,817, filed Jun. 4, 2010, the disclosure of which is incorporated by reference herein in its entirety.

### BACKGROUND

Previous studies have established that administration of the sPLA₂ inhibitor Amino-1,2-dioxoethyl)-2-ethyl-1-(phenylmethyl)-1H-indol-4-yl]oxy]acetic acid methyl ester (A-002), a prodrug of ((3-(2-Amino-1,2-dioxoethyl)-2-ethyl-1-(phenylmethyl)-1H-indol-4-yl)oxy)acetic acid (A-001), decreases lipid levels in humans. Importantly, these studies also established that co-administration of A-002 with other compounds used in the treatment of CVD such as statins or niacin drugs has a synergistic effect on lipid levels. In order to achieve the most efficient co-delivery of A-001/A-002 and other CVD therapeutics, it is desirable to incorporate both compounds into a single formulation, and even more desirable to incorporate both compounds into a single molecule. Provided herein are conjugate compounds that comprise A-001 or various derivatives thereof and one or more compounds used in the treatment of CVD linked together via a variety of chemical linkages. Also provided herein are formulations comprising these conjugate compounds and methods of using these conjugate compounds to alter lipid levels and treat various forms of CVD.

### DETAILED DESCRIPTION

The following description of the invention is merely intended to illustrate various embodiments of the invention. As such, the specific modifications discussed are not to be construed as limitations on the scope of the invention. It will be apparent to one skilled in the art that various equivalents, changes, and modifications may be made without departing from the scope of the invention, and it is understood that such equivalent embodiments are to be included herein.

Phospholipases A₂ are a class of enzymes that play a role in inflammation by hydrolyzing the sn-2 fatty acyl chain of glycerophospholipids to produce lysophospholipids, resulting in downstream production of arachidonic acid, prostaglandins, and leukotrienes. The classes of phospholipase A₂ in humans include secretory phospholipase A₂ (sPLA₂) types IB, IIA, ITC, IID, IIE, IIF, III, V, X, and XII, lipoprotein-associated phospholipase A₂ (Lp-PLA₂, also known as PLA₂ type VII), cytosolic phospholipase (cPLA₂), and calcium-independent phospholipase A₂ (iPLA₂). Elevated levels of sPLA₂ types IIA, IID, IIE, IIF, III, V, and X have been observed in all stages of atherosclerosis development and have been implicated in atherogenesis based on their ability to degrade phospholipid (Kimura-Matsumoto 2007). sPLA₂ type IIA has been found to be expressed at vascular smooth muscle cells and foam cells in human arteriosclerosis lesions, and this expression has been correlated to the development of arteriosclerosis (Menschikowski 1995; Elinder 1997; Hurt-Camejo 1997). Transgenic mice that express high levels of human type IIA sPLA₂ have increased LDL-C levels, decreased HDL levels, decreased LDL-C and HDL particle size, and exhibit arteriosclerotic lesions (Ivandic 1999; Tietge 2000), and develop arteriosclerosis at a higher rate compared to normal mice when given a high fat diet (Ivandic 1999). Treatment with sPLA₂ modifies LDL-C lipoproteins such that they have higher affinity for extracellular matrix proteins (Camejo 1998; Sartipy 1999; Hakala 2001), resulting in an increased retention of LDL-C particles in the arterial wall. sPLA₂ treatment also reduces approximately 50% of the phospholipid moiety of normal LDL-C, resulting in smaller and denser particles that are more likely to form non-soluble complexes with proteoglycans and glycosaminoglycans (Sartipy 1999). In addition, there is some evidence that sPLA₂ remodels HDL, resulting in HDL catabolism (Pruzanski 1998). sPLA₂ type V is present in atherosclerotic lesions associated with smooth muscle cells and in surrounding foam cells in lipid core areas of the plaque in mice and humans (Rosengren 2006). sPLA₂ type V has been shown to increase arteriosclerosis in mice, while a deficiency of sPLA₂ type V has been shown to reduce arteriosclerosis (Rosengren 2006; Bostrom 2007). Lp-PLA2 is highly expressed in the necrotic core of coronary lesions (Serruys 2008).

sPLA₂ expression has also been correlated with an increased risk of development of CAD. Higher circulating levels of sPLA₂, and of sPLA₂ type IIA specifically, have been observed in patients with documented CAD than in control patients (Kugiyama 1999; Liu 2003; Boekholdt 2005; Chait 2005; Hartford 2006). In addition, higher circulating levels of sPLA₂ were found to provide an accurate prognostic indicator for development of CAD in healthy individuals (Mallat 2007). Measurement of sPLA₂ activity has been shown to be an independent predictor of death and new or recurrent MI in subjects with ACS, and provides greater prognostic accuracy than measuring type IIA concentration only (Mallat 2005). It has also been proposed that sPLA₂ may have detrimental effects in the setting of ischemic events. This is based largely on the finding of sPLA₂ depositions in the necrotic center of infarcted human myocardium (Nijmeijer 2002).

Previous studies have established that once- or twice-daily administration of the sPLA₂ inhibitor prodrug A-002 decreases inflammatory marker levels, total cholesterol, LDL-C, total LDL-C particle, and small LDL-C particle levels in a stable cardiovascular disease (CVD) population, as well as in diabetic and high baseline LDL-C subpopulations (WO2008/137803). A-002 has the structure:

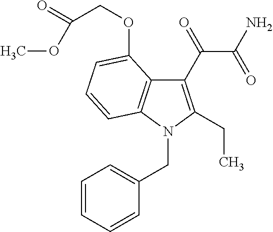

These previous studies also established that administration of A-002 in combination with compounds used in the treatment of CVD synergistically alters lipid levels (i.e., administration of the two compounds together alters lipid levels to a greater than the expected additive effect of the two compounds separately). For example, administration of A-002 in combination with one or more statins results in a synergistic decrease in LDL-C and small LDL-C particle levels in a stable CVD population, including in a high baseline LDL level subpopulation. This effect was not limited to a particular statin, but instead was observed across the entire spectrum of statins. Similarly, administration of A-002 in combination with one or more niacin drugs results in a synergistic decrease in TG levels and a synergistic increase in HDL levels. As with statins, this effect was not limited to a particular type of niacin drugs, but rather was observed across a spectrum of niacin drugs. In addition to these synergistic effects on lipid levels, previous studies have established that administration of A-002 in combination with statins results in a decrease in inflammatory markers levels and major adverse cardiac events in unstable subjects who have recently experienced an ACS event that is greater than the decrease obtained when either compound is administered alone.

A-002 is a prodrug of A-001 that is rapidly absorbed and hydrolyzed to A-001 following administration. A-001 has the structure:

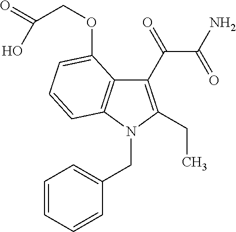

In all of studies discussed above, A-002 and the compounds used in the treatment of CVD were administered separately in tablet form. Although administration of separate compositions was effective at altering lipid levels, there is a need for more efficient compositions and methods for delivering these therapeutic combinations. In particular, there is a need for compositions and methods for delivery of both combination components in a single formulation, and even more preferably in a single conjugate compound. Such formulations and conjugate compounds are likely to increase patient compliance, and may have other therapeutic benefits as well (e.g., increased efficacy via increased bioavailability).

As disclosed herein, a series of conjugate compounds have been developed that combine A-001 or prodrugs, salts, solvates, or stereoisomers thereof with various compounds used in the treatment CVD in a single molecule. In certain of these embodiments, the conjugate compounds are conjugate prodrugs, meaning that the conjugate molecule is converted to two or more active molecules following administration.

In certain embodiments, the conjugate compounds provided herein may comprise A-001 and one or more niacin drugs. In these embodiments, the niacin drugs may be selected from the group consisting of, but not limited to, niacin/nicotinic acid, acipimox, and ERN (Niaspan®).

In certain embodiments, the conjugate compounds provided herein may comprise A-001 and one or more statins. In these embodiments, the statins may be selected from the group consisting of, but not limited to, atorvastatin or atorvastatin calcium (marketed as Lipitor® or Torvast®; see, e.g., U.S. Pat. No. 4,681,893 or 5,273,995), cerivastatin (marketed as Lipobay® or Baycol®), fluvastatin (marketed as Lescol®; U.S. Pat. No. 4,739,073), lovastatin (marketed as Mevacor® or Altocor®; see, e.g., U.S. Pat. No. 4,231,938), mevastatin, pitavastatin (marketed as Livalo® or Pitava®), pravastatin (marketed as Pravachol®, Mevalotin®, Selektine®, or Lipostat®; see, e.g., U.S. Pat. No. 4,346,227), rosuvastatin (marketed as Crestor®), and simvastatin (marketed as Zocor® or Lipex®; see, e.g., U.S. Pat. Nos. 4,444,784; 4,916,239; and 4,820,850), as well as various pharmaceutically acceptable salts, solvates, salts, stereoisomers, or nitroderivatives thereof.

In certain embodiments, the conjugate compounds disclosed herein may comprise one or more compounds used in the treatment of CVD other than statins or niacin drugs. In these embodiments, the compounds used in the treatment of CVD may be selected from the group consisting of, but not limited to, fibrates such as bezafibrate (Bezalip®), ciprofibrate (Modalim®), clofibrate, gemfibrozil (Lopid®), or fenofibrate (Antara®, TriCor®, ABT-335); bile acid sequestrants such as cholestyramine resin (Questran®, Prevalite®), colestipol hydrochloride (Colestid®), or colesevelam hydrochloride (WelChoi®, Cholestagel®); cholesterol absorption inhibitors such as ezetimibe (Zetia®), AVE 5530, or MD-0727; cholesteryl ester transfer protein (CETP) inhibitors such as JTT-705/RO4607381 (R1658), CP-529414 (Torcetrapib®), or MK-0859; microsomal triglyceride transfer protein (MTP) inhibitors such as AEGR-733; squalene synthase inhibitors such as lapaquistat acetate (TAK-475); ACE inhibitors such as lisinopril, captopril, enalapril, or nitrosated ACE inhibitors; angiotensin II receptor antagonists or nitrosated angiotensin II receptor antagonists; beta-adrenergic blockers or nitrosated beta-adrenergic blockers; calcium channel blockers; antithrombotics such as aspirin or nitrosated aspirin; and other miscellaneous compounds such as dextrothyroxine, ISIS 301012, cardioprotectants such as MC-1 antibody, glycoprotein IIb/IIIa inhibitors such as tirofiban hydrochloride (Aggrastat®), TG100-115, AEGR 773, AEGR 427, stanols, or sterols.

In certain embodiments of the conjugate compounds provided herein, the conjugate compounds may comprise an indole-based sPLA₂ inhibitor other than A-001. A variety of indole-based sPLA₂ inhibitors are known in the art. For example, indole-based sPLA₂ inhibitors that may be used in conjunction with the present invention include but are not limited to those set forth in U.S. Pat. Nos. 5,654,326 (Bach); 5,733,923 (Bach); 5,919,810 (Bach); 5,919,943 (Bach); 6,175,021 (Bach); 6,177,440 (Bach); 6,274,578 (Denney); and 6,433,001 (Bach), the entire disclosures of which are incorporated by reference herein. Methods of making indole-based sPLA₂ inhibitors are set forth in, for example, U.S. Pat. Nos. 5,986,106 (Khau); 6,265,591 (Anderson); and 6,380,397 (Anderson), the entire disclosures of which are incorporated by reference herein. sPLA₂ inhibitors for use in the present invention may be generated using these synthesis methods, or using any other synthesis method known in the art. In certain embodiments, sPLA₂ inhibitors for use in the present invention may be sPLA₂ type IIA, type V, and/or type X inhibitors. Various examples of indole-based sPLA₂ inhibitors are set forth below. These examples are merely provided as illustrations of the types of inhibitors that may be used in conjunction with the present invention, and as such are not meant to be limiting. One of ordinary skill in the art will recognize that a variety of other indole-based sPLA₂ inhibitors may be used.

In certain embodiments, sPLA₂ inhibitors for use in the current invention are 1H-indole-3-glyoxylamide compounds having the structure:

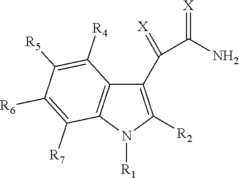

wherein:
each X is independently oxygen or sulfur;
R₁ is selected from the group consisting of (a), (b), and (c), wherein:
(a) is C₇-C₂₀ alkyl, C₇-C₂₀ alkenyl, C₇-C₂₀ alkynyl, carbocyclic radicals, or heterocyclic radicals;
(b) is a member of (a) substituted with one or more independently selected non-interfering substituents; and
(c) is the group -(L)-R₈₀, where, -(L)- is a divalent linking group of 1 to 12 atoms selected from carbon, hydrogen, oxygen, nitrogen, and sulfur, wherein the combination of atoms in -(L)- are selected from the group consisting of (i) carbon and hydrogen only, (ii) sulfur only, (iii) oxygen only, (iv) nitrogen and hydrogen only, (v) carbon, hydrogen, and sulfur only, and (vi) carbon, hydrogen, and oxygen only; and where R₈₀ is a group selected from (a) or (b); R₂ is hydrogen, halo, C₁-C₃ alkyl, C₃-C₄ cycloalkyl, C₃-C₄ cycloalkenyl, —O—(C₁-C₂ alkyl), —S—(C₁-C₂ alkyl), or a non-interfering substituent having a total of 1 to 3 atoms other than hydrogen; R₄ and R₅ are independently selected from the group consisting of hydrogen, a non-interfering substituent, and -(Lₐ)-(acidic group), wherein -(Lₐ)- is an acid linker having an acid linker length of 1 to 4; provided that at least one of R₄ and R₅ must be -(Lₐ)-(acidic group); R₆ and R₇ are each independently selected from hydrogen, non-interfering substituents, carbocyclic radicals, carbocyclic radicals substituted with non-interfering substituents, heterocyclic radicals, and heterocyclic radicals substituted with non-interfering substituents; provided that for any of the groups R₁, R₆, and R₇, the carbocyclic radical is selected from the group consisting of cycloalkyl, cycloalkenyl, phenyl, naphthyl, norbornanyl, bicycloheptadienyl, toluoyl, xylenyl, indenyl, stilbenzyl, terphenylyl, diphenylethylenyl, phenyl-cyclohexenly, acenaphthylenyl, and anthracenyl, biphenyl, bibenzylyl and related bibenzylyl homologues represented by the formula (bb),

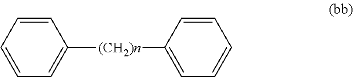

where n is a number from 1 to 8; provided, that for any of the groups R₁, R₆, and R₇, the heterocyclic radical is selected from the group consisting of pyrrolyl, furanyl, thiophenyl, pyrazolyl, imidazolyl, phenylimidazolyl, triazolyl, isoxazolyl, oxazolyl, thiazolyl, thiadiazolyl, indolyl, carbazolyl, norharmanyl, azaindolyl, benzofuranyl, dibenzofuranyl, thianaphtheneyl, dibenzothiophenyl, indazolyl, imidazo(1.2-A)pyridinyl, benzotriazolyl, anthranilyl, 1,2-benzisoxazolyl, benzoxazolyl, benzotriazolyl, purinyl, pryidinyl, dipyridylyl, phenylpyridinyl, benzylpyridinyl, pyrimidinyl, phenylpyrimidinyl, pyrazinyl, 1,3,5-triazinyl, quinolinyl, phthalazinyl, quinazolinyl, and quinoxalinyl; and
provided that for the groups R₁, R₂, R₄, R₅, R₆, and R₇ the non-interfering substituent is selected from the group consisting of C₁-C₆ alkyl, C₂-C₆ alkenyl, C₂-C₆ alkynyl, C₇-C₁₂ aralkyl, C₇-C₁₂ alkaryl, C₃-C₈ cycloalkyl, C₃-C₈ cycloalkenyl, phenyl, toluoyl, xylenyl, biphenyl, C₁-C₆ alkoxy, C₂-C₆ alkenyloxy, C₂-C₆ alkynyloxy, C₂-C₁₂ alkoxyalkyl, C₂-C₁₂ alkoxyalkyloxy, C₂-C₁₂ alkylcarbonyl, C₂-C₁₂ alkylcarbonylamino, C₂-C₁₂ alkoxyamino, C₂-C₁₂ alkoxyaminocarbonyl, C₂-C₁₂ alkylamino, C₁-C₆ alkylthio, C₂-C₁₂ alkylthiocarbonyl, C₁-C₆ alkylsulfinyl, C₁-C₆ alkylsulfonyl, C₂-C₆ haloalkoxy, C₁-C₆ haloalkylsulfonyl, C₂-C₆ haloalkyl, C₁-C₆ hydroxyalkyl, —C(O)O(C₁-C₆ alkyl), —(CH₂), —O—(C₁-C₆ alkyl), benzyloxy, phenoxy, phenylthio, —(CONHSO₂R), —CHO, amino, amidino, bromo, carbamyl, carboxyl, carbalkoxy, —(CH₂)ₙ—CO₂H, chloro, cyano, cyanoguanidinyl, fluoro, guanidino, hydrazide, hydrazino, hydrazido, hydroxy, hydroxyamino, iodo, nitro, phosphono, —SO₃H, thioacetal, thiocarbonyl, and C₁-C₆ carbonyl, where n is from 1 to 8;
and pharmaceutically acceptable salts, solvates, prodrug derivatives, racemates, tautomers, or optical isomers thereof.

In certain of these embodiments, -(L)- has the formula:

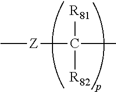

wherein R₈₁ and R₈₂ are each independently selected from the group consisting of hydrogen, C₁-C₁₀ alkyl, carboxy, carbalkoxy, and halo; p is a number from 1 to 5; and Z is selected from the group consisting of a bond, —(CH₂)—, —O—, —N(C₁-C₁₀ alkyl)- , —NH—, and —S—.

In certain of these embodiments wherein R₄ is -(Lₐ)-(acidic group), the acid linker -(Lₐ)- has the formula:

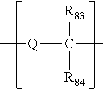

wherein Q is selected from the group consisting of —(CH₂)—, —O—, —NH—, and —S—; and R₈₃ and R₈₄ are each independently selected from the group consisting of hydrogen, C₁-C₁₀ alkyl, aryl, C₁-C₁₀ alkaryl, C₁-C₁₀ aralkyl, hydroxy, and halo.

In certain of these embodiments wherein R₅ is -(Lₐ)-(acidic group), the acid linker -(Lₐ)- has the formula:

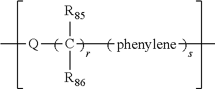

wherein r is a number from 2 to 7; s is 0 or 1; Q is selected from the group consisting of —(CH₂)—, —O—, —NH—, and —S—; and R₈₅ and R₈₆ are each independently selected from the group consisting of hydrogen, C₁-C₁₀ alkyl, aryl, C₁-C₁₀ alkaryl, C₁-C₁₀ aralkyl, carboxy, carbalkoxy, and halo.

In certain embodiments, a 1H-indole-3-glyoxylamide compound for use in the present invention is selected from the group consisting of: ((3-(2-Amino-1,2-dioxoethyl)-2-ethyl-1-(phenylmethyl)-1H-indol-4-yl)oxy)acetic acid; [[3-(2-Amino-1,2-dioxoethyl)-2-ethyl-1-(phenylmethyl)-1H-indol-4-yl]oxy]acetic acid methyl ester; ((3-(2-Amino-1,2-dioxoethyl)-2-methyl-1-(phenylmethyl)-1H-indol-4-yl)oxy)acetic acid; d1-2-((3-(2-Amino-1,2-dioxoethyl)-2-methyl-1-(phenylmethyl)-1H-indol-4-yl)oxy)propanoic acid; ((3-(2-Amino-1,2-dioxoethyl)-1-((1,1′-biphenyl)-2-ylmethyl)-2-methyl-1H-indol-4-yl)oxy)acetic acid; ((3-(2-Amino-1,2-dioxoethyl)-1-((1,1′-biphenyl)-3-ylmethyl)-2-methyl-1H-indol-4-yl)oxy)acetic acid; ((3-(2-Amino-1,2-dioxoethyl)-1-((1,1′-biphenyl)-4-ylmethyl)-2-methyl-1H-indol-4-yl)oxy)acetic acid; ((3-(2-Amino-1,2-dioxoethyl)-1-((2,6-dichlorophenyl)methyl)-2-methyl-1H-indol-4-yl)oxy)acetic acid; ((3-(2-Amino-1,2-dioxoethyl)-1-(4(-fluorophenyl)methyl)-2-methyl-1H-indol-4-yl)oxy)acetic acid; ((3-(2-Amino-1,2-dioxoethyl)-2-methyl-1-((1-naphthalenyl)methyl)-1H-indol-4-yl)oxy)acetic acid; ((3-(2-Amino-1,2-dioxoethyl)-1-((3-chlorophenyl)methyl)-2-ethyl-1H-indol-4-yl)oxy)acetic acid; ((3-(2-Amino-1,2-dioxoethyl)-1-((1,1′-biphenyl)-2-ylmethyl)-2-ethyl-1H-indol-4-yl)oxy)acetic acid; ((3-(2-amino-1,2-dioxoethyl)-1-((1,1′-biphenyl)-2-ylmethyl)-2-propyl-1H-indol-4-yl)oxy)acetic acid; ((3-(2-Amino-1,2-dioxoethyl)-2-cyclopropyl-1-(phenylmethyl)-1H-indol-4-yl) oxy)acetic acid; ((3-(2-Amino-1,2-dioxoethyl)-1-((1,1′-biphenyl)-2-ylmethyl)-2-cyclopropyl-1H-indol-4-yl)oxy)acetic acid; and 4-((3-(2-Amino-1,2-dioxoethyl)-2-ethyl-1-(phenylmethyl)-1H-indol-5-yl)oxy)butanoic acid, or pharmaceutically acceptable salts, solvates, prodrug derivatives, racemates, tautomers, or optical isomers thereof.

In certain embodiments, sPLA₂ inhibitors for use in the current invention are 1H-indole-3-glyoxylamide compounds having the structure:

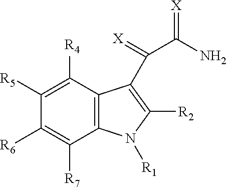

wherein:
both X are oxygen;
R₁ is selected from the group consisting of:

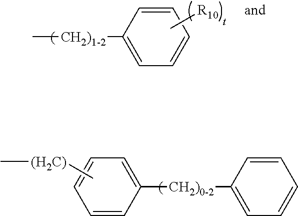

wherein R₁₀ is a radical independently selected from halo, C₁-C₁₀ alkoxy, —S—(C₁-C₁₀ alkyl), and C₁-C₁₀ haloalkyl, and t is a number from 0 to 5;
R₂ is selected from the group consisting of halo, cyclopropyl, methyl, ethyl, and propyl;
R₄ and R₅ are independently selected from the group consisting of hydrogen, a non-interfering substituent, and -(Lₐ)-(acidic group), wherein -(Lₐ)- is an acid linker; provided that the acid linker -(Lₐ)- for R₄ is selected from the group consisting of:

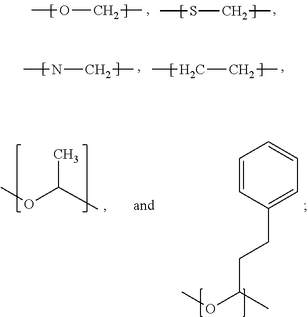

and provided that the acid linker -(Lₐ)- for R₅ is selected from the group consisting of:

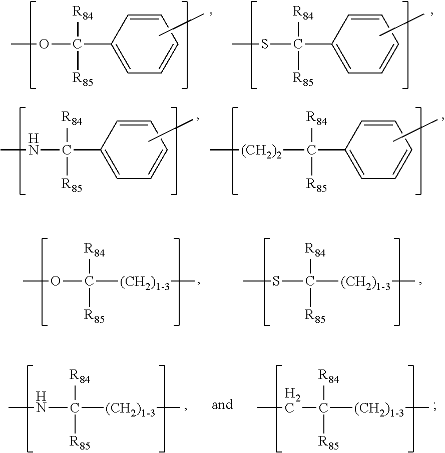

wherein R₈₄ and R₈₅ are each independently selected from the group consisting of hydrogen, C₁-C₁₀ alkyl, aryl, C₁-C₁₀ alkaryl, C₁-C₁₀ aralkyl, carboxy, carbalkoxy, and halo; provided that at least one of R₄ and R₅ must be -(Lₐ)-(acidic group), and (acidic group) on -(Lₐ)-(acidic group) of R₄ or R₅ is selected from —CO₂H, —SO₃H, or —P(O)(OH)₂;
R₆ and R₇ are each independently selected from the group consisting of hydrogen and non-interfering substituents, with the non-interfering substituents being selected from the group consisting of: C₁-C₆ alkyl, C₂-C₆ alkenyl, C₂-C₆ alkynyl, C₇-C₁₂ aralkyl, C₇-C₁₂ alkaryl, C₃-C₈ cycloalkyl, C₃-C₈ cycloalkenyl, phenyl, toluoyl, xylenyl, biphenyl, C₁-C₆ alkoxy, C₂-C_{alkenyloxy, C}₂-C₆ alkynyloxy, C₂-C₁₂ alkoxyalkyl, C₂-C₁₂ alkoxyalkyloxy, C₂-C₁₂ alkylcarbonyl, C₂-C₁₂ alkylcarbonylamino, C₂-C₁₂ alkoxyamino, C₂-C₁₂ alkoxyaminocarbonyl, C₂-C₁₂ alkylamino, C₁-C₆ alkylthio, C₂-C₁₂ alkylthiocarbonyl, C₁-C₆ alkylsulfinyl, C₁-C₆ alkylsulfonyl, C₂-C₆ haloalkoxy, C₁-C₆ haloalkylsulfonyl, C₂-C₆ haloalkyl, C₁-C₆ hydroxyalkyl, —C(O)O(C₁-C₆ alkyl), —(CH₂), —O—(C₁-C₆ alkyl), benzyloxy, phenoxy, phenylthio, —(CONHSO₂R), —CHO, amino, amidino, bromo, carbamyl, carboxyl, carbalkoxy, —(CH₂)ₙ—CO₂H, chloro, cyano, cyanoguanidinyl, fluoro, guanidino, hydrazide, hydrazino, hydrazido, hydroxy, hydroxyamino, iodo, nitro, phosphono, —SO₃H, thioacetal, thiocarbonyl, and C₁-C₆ carbonyl; wherein n is from 1 to 8;
and pharmaceutically acceptable salts, solvates, prodrug derivatives, racemates, tautomers, or optical isomers thereof.

In certain embodiments, 1H-indole-3-glyoxylamide compounds for use in the present invention are selected from the group consisting of: ((3-(2-Amino-1,2-dioxoethyl)-2-methyl-1-(phenylmethyl)-1H-indol-4-yl)oxy)acetic acid; ((3-(2-Amino-1,2-dioxoethyl)-2-methyl-1-(phenylmethyl)-1H-indol-4-yl)oxy)acetic acid methyl ester; d1-2-((3-(2-Amino-1,2-dioxoethyl)-2-methyl-1-(phenylmethyl)-1H-indol-4-yl) oxy)propanoic acid; d1-2-((3-(2-Amino-1,2-dioxoethyl)-2-methyl-1-(phenylmethyl)-1H-indol-4-yl)oxy)propanoic acid methyl ester; ((3-(2-Amino-1,2-dioxoethyl)-1-((1,1′-biphenyl)-2-ylmethyl)-2-methyl-1H-indol-4-yl)oxy)acetic acid; ((3-(2-Amino-1,2-dioxoethyl)-1-((1,1′-biphenyl)-2-ylmethyl)-2-methyl-1H-indol-4-yl)oxy)acetic acid methyl ester; ((3-(2-Amino-1,2-dioxoethyl)-1-((1,1′-biphenyl)-3-ylmethyl)-2-methyl-1H-indol-4-yl)oxy)acetic acid; ((3-(2-Amino-1,2-dioxoethyl)-1-((1,1′-biphenyl)-3-ylmethyl)-2-methyl-1H-indol-4-yl)oxy)acetic acid methyl ester; ((3-(2-Amino-1,2-dioxoethyl)-1-((1,1′-biphenyl)-4-ylmethyl)-2-methyl-1H-indol-4-yl)oxy)acetic acid; ((3-(2-Amino-1,2-dioxoethyl)-1-((1,1′-biphenyl)-4-ylmethyl)-2-methyl-1H-indol-4-yl)oxy)acetic acid methyl ester; ((3-(2-Amino-1,2-dioxoethyl)-1-((2,6-dichlorophenyl)methyl)-2-methyl-1H-indol-4-yl)oxy)acetic acid; ((3-(2-Amino-1,2-dioxoethyl)-1-((2,6-dichlorophenyl)methyl)-2-methyl-1H-indol-4-yl)oxy)acetic acid methyl ester; 43-(2-Amino-1,2-dioxoethyl)-1-(4(-fluorophenyl)methyl)-2-methyl-1H-indol-4-yl)oxy)acetic acid; ((3-(2-Amino-1,2-dioxoethyl)-1-(4(-fluorophenyl)methyl)-2-methyl-1H-indol-4-yl)oxy)acetic acid methyl ester; 43-(2-Amino-1,2-dioxoethyl)-2-methyl-1-((1-naphthalenyl)methyl)-1H-indol-4-yl)oxy)acetic acid; ((3-(2-Amino-1,2-dioxoethyl)-2-methyl-1-((1-naphthalenyl)methyl)-1H-indol-4-yl)oxy)acetic acid methyl ester; ((3-(2-Amino-1,2-dioxoethyl)-1-((3-chlorophenyl)methyl)-2-ethyl-1H-indol-4-yl)oxy)acetic acid; ((3-(2-Amino-1,2-dioxoethyl)-1-((3-chlorophenyl)methyl)-2-ethyl-1H-indol-4-yl)oxy)acetic acid methyl ester; ((3-(2-Amino-1,2-dioxoethyl)-1-((1,1′-biphenyl)-2-ylmethyl)-2-ethyl-1H-indol-4-yl)oxy)acetic acid; ((3-(2-Amino-1,2-dioxoethyl)-1-((1,1′-biphenyl)-2-ylmethyl)-2-ethyl-1H-indol-4-yl)oxy)acetic acid methyl ester; ((3-(2-amino-1,2-dioxoethyl)-1-((1,1′-biphenyl)-2-ylmethyl)-2-propyl-1H-indol-4-yl)oxy)acetic acid; ((3-(2-amino-1,2-dioxoethyl)-1-((1,1′-biphenyl)-2-ylmethyl)-2-propyl-1H-indol-4-yl)oxy)acetic acid methyl ester; ((3-(2-Amino-1,2-dioxoethyl)-2-cyclopropyl-1-(phenylmethyl)-1H-indol-4-yl)oxy)acetic acid; ((3-(2-Amino-1,2-dioxoethyl)-2-cyclopropyl-1-(phenylmethyl)-1H-indol-4-yl)oxy)acetic acid methyl ester; ((3-(2-Amino-1,2-dioxoethyl)-1-((1,1′-biphenyl)-2-ylmethyl)-2-cyclopropyl-1H-indol-4-yl)oxy)acetic acid; ((3-(2-Amino-1,2-dioxoethyl)-1-((1,1′-biphenyl)-2-ylmethyl)-2-cyclopropyl-1H-indol-4-yl)oxy)acetic acid methyl ester; 4-((3-(2-Amino-1,2-dioxoethyl)-2-ethyl-1-(phenylmethyl)-1H-indol-5-yl)oxy)butanoic acid; 4-((3-(2-Amino-1,2-dioxoethyl)-2-ethyl-1-(phenylmethyl)-1H-indol-5-yl)oxy)butanoic acid tert-butyl ester, or pharmaceutically acceptable salts, solvates, prodrug derivatives, racemates, tautomers, or optical isomers thereof.

In certain embodiments, sPLA₂ inhibitors for use in the current invention are 1H-indole-3-glyoxylamide compounds having the structure:

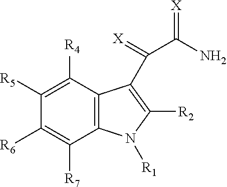

wherein:
each X is independently oxygen or sulfur;
R₁ is selected from groups (a), (b), and (c) wherein:

(a) is C₇-C₂₀ alkyl, C₇-C₂₀ alkenyl, C₇-C₂₀ alkynyl, carbocyclic radical, or heterocyclic radical;

(b) is a member of (a) substituted with one or more independently selected non-interfering substituents; and

(c) is the group -(L)-R₈₀, wherein -(L)- is a divalent linking group of 1 to 12 atoms selected from carbon, hydrogen, oxygen, nitrogen, and sulfur; wherein the combination of atoms in -(L)- are selected from the group consisting of (i) carbon and hydrogen only, (ii) sulfur only, (iii) oxygen only, (iv) nitrogen and hydrogen only, (v) carbon, hydrogen, and sulfur only, and (vi) and carbon, hydrogen, and oxygen only; and where R₈₀ is a group selected from (a) or (b); R₂ is selected from the group consisting of hydrogen, halo, C₁-C₃ alkyl, C₃-C₄ cycloalkyl, C₃-C₄ cycloalkenyl, —O—(C₁-C₂ alkyl), —S—(C₁-C₂ alkyl), and a non-interfering substituent having a total of 1 to 3 atoms other than hydrogen;

R₄ and R₅ are independently selected from the group consisting of hydrogen, a non-interfering substituent, and the group -(Lₐ)-(acidic group), wherein -(Lₐ)- is an acid linker having an acid linker length of 1 to 4; provided that at least one of R₄ and R₅ is -(Lₐ)-(acidic group);
R₆ and R₇ are each independently selected from the group consisting of hydrogen, non-interfering substituents, carbocyclic radicals, carbocyclic radicals substituted with non-interfering substituents, heterocyclic radicals, and heterocyclic radicals substituted with non-interfering substituents;
and pharmaceutically acceptable salts, solvates, prodrug derivatives, racemates, tautomers, or optical isomers thereof.

In certain embodiments, sPLA₂ inhibitors for use in the current invention are methyl ester prodrug derivatives of 1H-indole-3-glyoxylamide compounds having the structure:

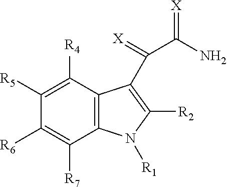

wherein:
both X are oxygen;
R₁ is selected from the group consisting of:

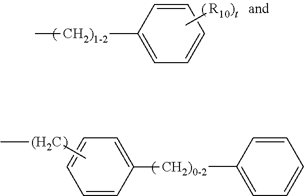

wherein R₁₀ is a radical independently selected from halo, C₁-C₁₀ alkyl, C₁-C₁₀ alkoxy, —S—(C₁-C₁₀ alkyl), and C₁-C₁₀ haloalkyl, and t is a number from 0 to 5;

R₂ is selected from the group consisting of halo, cyclopropyl, methyl, ethyl, and propyl;

R₄ and R₅ are independently selected from the group consisting of hydrogen, a non-interfering substituent, and -(Lₐ)-(acidic group), wherein -(Lₐ)- is an acid linker; provided that the acid linker -(Lₐ)- for R₄ is selected from the group consisting of:

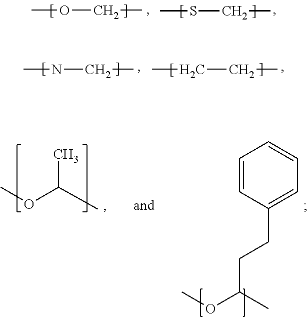

and provided that the acid linker -(Lₐ)- for R₅ is selected from the group consisting of:

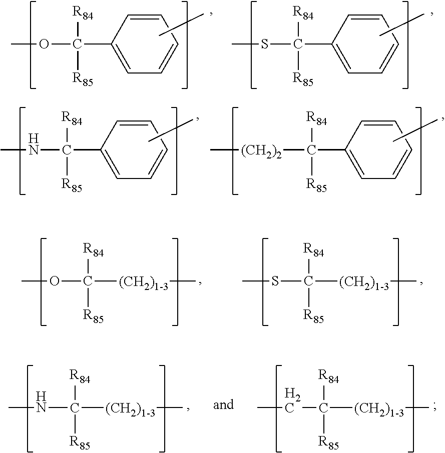

wherein R₈₄ and R₈₅ are each independently selected from the group consisting of hydrogen, C₁-C₁₀ alkyl, aryl, C₁-C₁₀ alkaryl, C₁-C₁₀ aralkyl, carboxy, carbalkoxy, and halo; provided that at least one of R₄ and R₅ must be -(Lₐ)-(acidic group), and (acidic group) on -(Lₐ)-(acidic group) of R₄ or R₅ is selected from —CO₂H, —SO₃H, or —P(O)(OH)₂;
R₆ and R₇ are each independently selected from the group consisting of hydrogen and non-interfering substituents, with the non-interfering substituents being selected from the group consisting of: C₁-C₆ alkyl, C₂-C₆ alkenyl, C₂-C₆ alkynyl, C₇-C₁₂ aralkyl, C₇-C₁₂ alkaryl, C₃-C₈ cycloalkyl, C₃-C₈ cycloalkenyl, phenyl, toluoyl, xylenyl, biphenyl, C₁-C₆ alkoxy, C₂-C₆ alkenyloxy, C₂-C₆ alkynyloxy, C₂-C₁₂ alkoxyalkyl, C₂-C₁₂ alkoxyalkyloxy, C₂-C₁₂ alkylcarbonyl, C₂-C₁₂ alkylcarbonylamino, C₂-C₁₂ alkoxyamino, C₂-C₁₂ alkoxyaminocarbonyl, C₂-C₁₂ alkylamino, C₁-C₆ alkylthio, C₂-C₁₂ alkylthiocarbonyl, C₁-C₆ alkylsulfinyl, C₁-C₆ alkylsulfonyl, C₂-C₆ haloalkoxy, C₁-C₆ haloalkylsulfonyl, C₂-C₆ haloalkyl, C₁-C_{hydroxyalkyl, —C(O)O(C}₁-C₆ alkyl), —(CH₂), —O—(C₁-C₆ alkyl), benzyloxy, phenoxy, phenylthio, —(CONHSO₂R), —CHO, amino, amidino, bromo, carbamyl, carboxyl, carbalkoxy, —(CH₂)ₙ—CO₂H, chloro, cyano, cyanoguanidinyl, fluoro, guanidino, hydrazide, hydrazino, hydrazido, hydroxy, hydroxyamino, iodo, nitro, phosphono, —SO₃H, thioacetal, thiocarbonyl, and C₁-C₆ carbonyl; wherein n is from 1 to 8;
and pharmaceutically acceptable salts, solvates, prodrug derivatives, racemates, tautomers, or optical isomers thereof.

In certain embodiments, sPLA₂ inhibitors for use in the current invention are (acyloxy) alkyl ester prodrug derivatives of 1H-indole-3-glyoxylamide compounds having the structure:

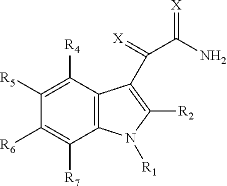

wherein:
both X are oxygen;
R₁ is selected from the group consisting of:

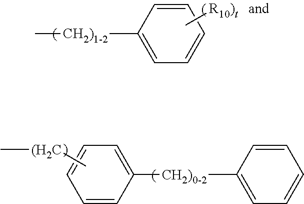

wherein R₁₀ is a radical independently selected from halo, C₁-C₁₀ alkyl, C₁-C₁₀ alkoxy, —S—(C₁-C₁₀ alkyl), and C₁-C₁₀ haloalkyl, and t is a number from 0 to 5;
R₂ is selected from the group consisting of halo, cyclopropyl, methyl, ethyl, and propyl;
R₄ and R₅ are independently selected from the group consisting of hydrogen, a non-interfering substituent, and -(Lₐ)-(acidic group), wherein -(Lₐ)- is an acid linker; provided that the acid linker -(Lₐ)- for R₄ is selected from the group consisting of:

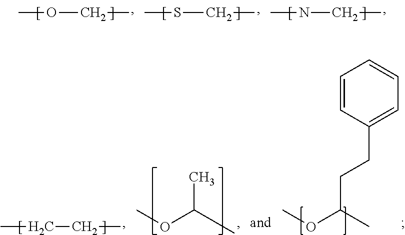

and provided that the acid linker -(Lₐ)- for R₅ is selected from the group consisting of:

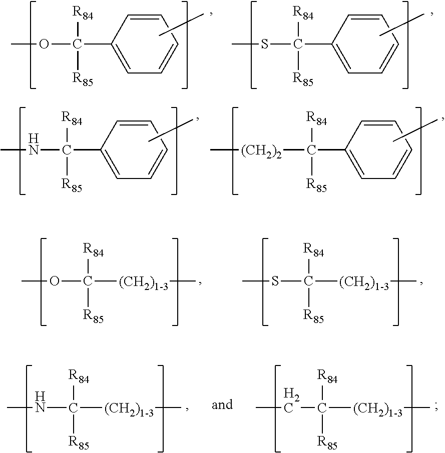

wherein R₈₄ and R₈₅ are each independently selected from the group consisting of hydrogen, C₁-C₁₀ alkyl, aryl, C₁-C₁₀ alkaryl, C₁-C₁₀ aralkyl, carboxy, carbalkoxy, and halo; provided that at least one of R₄ and R₅ must be -(Lₐ)-(acidic group), and (acidic group) on -(Lₐ)-(acidic group) of R₄ or R₅ is selected from —CO₂H, —SO₃H, or —P(O)(OH)₂;
R₆ and R₇ are each independently selected from the group consisting of hydrogen and non-interfering substituents, with the non-interfering substituents being selected from the group consisting of: C₁-C₆ alkyl, C₂-C₆ alkenyl, C₂-C₆ alkynyl, C₇-C₁₂ aralkyl, C₇-C₁₂ alkaryl, C₃-C₈ cycloalkyl, C₃-C₈ cycloalkenyl, phenyl, toluoyl, xylenyl, biphenyl, C₁-C₆ alkoxy, C₂-C₆ alkenyloxy, C₂-C₆ alkynyloxy, C₂-C₁₂ alkoxyalkyl, C₂-C₁₂ alkoxyalkyloxy, C₂-C₁₂ alkylcarbonyl, C₂-C₁₂ alkylcarbonylamino, C₂-C₁₂ alkoxyamino, C₂-C₁₂ alkoxyaminocarbonyl, C₂-C₁₂ alkylamino, C₁-C₆ alkylthio, C₂-C₁₂ alkylthiocarbonyl, C₁-C₆ alkylsulfinyl, C₁-C₆ alkylsulfonyl, C₂-C₆ haloalkoxy, C₁-C₆ haloalkylsulfonyl, C₂-C₆ haloalkyl, C₁-C₆ hydroxyalkyl, —C(O)O(C₁-C₆ alkyl), —(CH₂), —O—(C₁-C₆ alkyl), benzyloxy, phenoxy, phenylthio, —(CONHSO₂R), —CHO, amino, amidino, bromo, carbamyl, carboxyl, carbalkoxy, —(CH₂)ₙ—CO₂H, chloro, cyano, cyanoguanidinyl, fluoro, guanidino, hydrazide, hydrazino, hydrazido, hydroxy, hydroxyamino, iodo, nitro, phosphono, —SO₃H, thioacetal, thiocarbonyl, and C₁-C₆ carbonyl; wherein n is from 1 to 8;
and pharmaceutically acceptable salts, solvates, prodrug derivatives, racemates, tautomers, or optical isomers thereof.

In certain embodiments, sPLA₂ inhibitors for use in the current invention are substituted tricyclics having the structure:

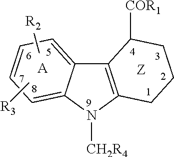

wherein:
R₁ is selected from the group consisting of —NHNH₂ and —NH₂;
R₂ is selected from the group consisting of —OH and —O(CH₂)ₘR₅; wherein R₅ is selected from the group consisting of H, —CO₂H, —CO₂(C₁-C₄ alkyl), —SO₃H, —SO₃(C₁-C₄ alkyl), tetrazolyl, —CN, —NH₂, —NHSO₂R₁₅, —CONHSO₂R₁₅, phenyl, phenyl substituted with —CO₂H or —CO₂(C₁-C₄)alkyl, and

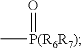

wherein R₆ and R₇ are each independently selected from the group consisting of —OH, —O(C₁-C₄)alkyl; R₁₅ is selected from the group consisting of —(C₁-C₆)alkyl and —CF₃; and m is 1-3; R₃ is selected from the group consisting of H, —O(C₁-C₄)alkyl, halo, —(C₁-C₆)alkyl, phenyl, —(C₁-C₄)alkylphenyl, phenyl substituted with —(C₁-C₆)alkyl, halo, or —CF₃, —CH₂OSi(C₁-C₆)alkyl, furyl, thiophenyl, —(C₁-C₆)hydroxyalkyl, and —(CH₂)ₙR₈; wherein R₈ is selected from the group consisting of H, —CONH₂, —NR₉R₁₀, —CN, and phenyl; wherein R₉ and R₁₀ are each independently —(C₁-C₄)alkyl or -phenyl(C₁-C₄)alkyl; and n is 1 to 8;
R₄ is selected from the group consisting of H, —(C₅-C₁₄)alkyl, —(C₃-C₁₄)cycloalkyl, pyridyl, phenyl, and phenyl substituted with —(C₁-C₆)alkyl, halo, —CF₃, —OCF₃, —(C₁-C₄)alkoxy, —CN, —(C₁-C₄)alkylthio, phenyl(C₁-C₄)alkyl, —(C₁-C₄)alkylphenyl, phenyl, phenoxy, or naphthyl;
A is selected from the group consisting of phenyl and pyridyl wherein the nitrogen is at the 5-, 6-, 7-, or 8-position;
Z is selected from the group consisting of cyclohexenyl, phenyl, pyridyl wherein the nitrogen is at the 1-, 2-, or 3-position, and a 6-membered heterocyclic ring having one heteroatom selected from the group consisting of sulfur and oxygen at the 1-, 2-, or 3-position and nitrogen at the 1-, 2-, 3-, or 4- position, or wherein one carbon on the heterocyclic ring is optionally substituted with ═O; and wherein one of A or Z is a heterocyclic ring;
and pharmaceutically acceptable salts, solvates, prodrug derivatives, racemates, tautomers, or optical isomers thereof.

In certain embodiments, sPLA₂ inhibitors for use in the current invention are substituted tricyclics having the structure:

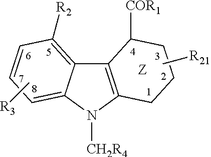

wherein:
Z is selected from the group consisting of cyclohexenyl and phenyl;
R₂₁ is a non-interfering substituent;

### R₁ is —NHNH₂ or —NH₂;

R₂ is selected from the group consisting of —OH and —O(CH₂)ₘR₅; wherein R₅ is selected from the group consisting of H, —CO₂H, —CONH₂, —CO₂(C₁-C₄ alkyl), —SO₃H, —SO₃(C₁-C₄ alkyl), tetrazolyl, —CN, —NH₂, —NHSO₂R₁₅, —CONHSO₂R₁₅, phenyl, phenyl substituted with —CO₂H or —CO₂(C₁-C₄)alkyl, and

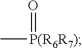

wherein R₆ and R₇ are each independently selected from the group consisting of —OH, —O(C₁-C₄)alkyl; R₁₅ is selected from the group consisting of —(C₁-C₆)alkyl and —CF₃; and m is 1-3;
R₃ selected from the group consisting of H, —O(C₁-C₄)alkyl, halo, —(C₁-C₆)alkyl, phenyl, —(C₁-C₄)alkylphenyl, phenyl substituted with —(C₁-C₆)alkyl, halo, or —CF₃, —CH₂OSi(C₁-C₆)alkyl, furyl, thiophenyl, —(C₁-C₆)hydroxyalkyl, and —(CH₂)ₙR₈; wherein R₈ is selected from the group consisting of H, —CONH₂, —NR₉R₁₀, —CN, and phenyl; R₉ and R₁₀ are each independently selected from the group consisting of H, —CF₃, phenyl, —(C₁-C₄)alkyl, —(C₁-C₄)alkylphenyl, and -phenyl(C₁-C₄)alkyl; and n is 1 to 8;
R₄ is selected from the group consisting of H, —(C₅-C₁₄)alkyl, —(C₃-C₁₄)cycloalkyl, pyridyl, phenyl, phenyl substituted with —(C₁-C₆)alkyl, halo, —CF₃, —OCF₃, —(C₁-C₄)alkoxy, —CN, —(C₁-C₄)alkylthio, -phenyl(C₁-C₄)alkyl, —(C₁-C₄)alkylphenyl, phenyl, phenoxy and naphthyl;
and pharmaceutically acceptable salts, solvates, prodrug derivatives, racemates, tautomers, or optical isomers thereof.

In certain embodiments, sPLA₂ inhibitors for use in the current invention are selected from the group consisting of: {9-[(phenyl)methyl]-5-carbamoylcarbazol-4-yl}oxyacetic acid; 9-benzyl-5,7-dimethoxy-1,2,3,4-tetrahydrocarbazole-4-carboxylic acid hydrazide; 9-benzyl-5,7-dimethoxy-1,2,3,4-tetrahydrocarbazole-4-carboxamide; [9-benzyl-4-carbamoyl-7-methoxy-1,2,3,4-tetrahydrocarbazol-5-yl]oxyacetic acid; [9-benzyl-4-carbamoyl-7-methoxycarbazol-5-yl]oxyacetic acid; methyl[9-benzyl-4-carbamoyl-7-methoxycarbazol-5-yl]oxyacetic acid; 9-benzyl-7-methoxy-5-cyanomethyloxy-1,2,3,4-tetrahydrocarbazole-4-carboxamide; 9-benzyl-7-methoxy-5-(1H-tetrazol-5-yl-methyl)oxy)-1,2,3,4-tetrahydrocarbazole-4-carboxamide; {9-[(phenyl)methyl]-5-carbamoyl-2-methyl-carbazol-4-yl}oxyacetic acid; {9-[(3-fluorophenyl)methyl]-5-carbamoyl-2-methylcarbazol-4-yl}oxyacetic acid; {9-[(3-methylphenyl)methyl]-5-carbamoyl-2-methylcarbazol-4-yl}oxyacetic acid; {9-[(phenyl)methyl]-5-carbamoyl-2-(4-trifluoromethylphenyl)-carbazol-4-yl}oxyacetic acid; 9-benzyl-5-(2-methanesulfonamido)ethyloxy-7-methoxy-1,2,3,4-tetrahydrocarbazole-4-carboxamide; 9-benzyl-4-(2-methanesulfonamido)ethyloxy-2-methoxycarbazole-5-carboxamide; 9-benzyl-4-(2-trifluoromethanesulfonamido)ethyloxy-2-methoxycarbazole-5-carboxamide; 9-benzyl-5-methanesulfonamidoylmethyloxy-7-methoxy-1,2,3,4-tetrahydrocarbazole-4-carboxamide; 9-benzyl-4-methanesulfonamidoylmethyloxy-carbazole-5-carboxamide; [5-carbamoyl-2-pentyl-9-(phenylmethyl)carbazol-4-yl]oxyacetic acid; [5-carbamoyl-2-(1-methylethyl)-9-(phenylmethyl)carbazol-4-yl]oxyacetic acid; [5-carbamoyl-9-(phenylmethyl)-2-[(tri(-1-methylethyl)silyl)oxymethyl]carbazol-4-yl]oxyacetic acid; [5-carbamoyl-2-phenyl-9-(phenylmethyl)carbazol-4-yl]oxyacetic acid; [5-carbamoyl-2-(4-chlorophenyl)-9-(phenylmethyl)carbazol-4-yl]oxyacetic acid; [5-carbamoyl-2-(2-furyl)-9-(phenylmethyl)carbazol-4-yl]oxyacetic acid; [5-carbamoyl-9-(phenylmethyl)-2-[(tri(−1-methylethyl)silyl)oxymethyl]carbazol-4-yl]oxyacetic acid; {9-[(2-Fluorophenyl)methyl]-5-carbamoylcarbazol-4-yl}oxyacetic acid; {9-[(2-trifluoromethylphenyl)methyl]-5-carbamoylcarbazol-4-yl}oxyacetic acid; {9-[(2-benzylphenyl)methyl]-5-carbamoylcarbazol-4-yl}oxyacetic acid; {9-[(1-naphthyl)methyl]-5-carbamoylcarbazol-4-yl}oxyacetic acid; {9-[(2-cyanophenyl)methyl]-5-carbamoylcarbazol-4-yl}oxyacetic acid; {9-[(3-cyanophenyl)methyl]-5-carbamoylcarbazol-4-yl}oxyacetic acid; {9-[(3,5-dimethylphenyl)methyl]-5-carbamoylcarbazol-4-yl}oxyacetic acid; {9-[(3-iodophenyl)methyl]-5-carbamoylcarbazol-4-yl}oxyacetic acid; {9-[(2-Chlorophenyl)methyl]-5-carbamoylcarbazol-4-yl}oxyacetic acid; {9-[(2,3-difluorophenyl)methyl]-5-carbamoylcarbazol-4-yl}oxyacetic acid; {9-[(2,6-difluorophenyl)methyl]-5-carbamoylcarbazol-4-yl}oxyacetic acid; {9-[(2,6-dichlorophenyl)methyl]-5-carbamoylcarbazol-4-yl}oxyacetic acid; {9-[(2-biphenyl)methyl]-5-carbamoylcarbazol-4-yl}oxyacetic acid; {9-[(2-Biphenyl)methyl]-5-carbamoylcarbazol-4-yl}oxyacetic acid methyl ester; [9-Benzyl-4-carbamoyl-1,2,3,4-tetrahydrocarbazol-5-yl]oxyacetic acid; {9-[(2-Pyridyl)methyl]-5-carbamoylcarbazol-4-yl}oxyacetic acid; {9-[(3-Pyridyl)methyl]-5-carbamoylcarbazol-4-yl}oxyacetic acid; [9-benzyl-4-carbamoyl-8-methyl-1,2,3,4-tetrahydrocarbazol-5-yl]oxyacetic acid; [9-benzyl-5-carbamoyl-1-methylcarbazol-4-yl]oxyacetic acid; [9-benzyl-4-carbamoyl-8-fluoro-1,2,3,4-tetrahydrocarbazol-5-yl]oxyacetic acid; [9-benzyl-4-carbamoyl-8-chloro-1,2,3,4-tetrahydrocarbazol-5-yl]oxyacetic acid; [5-carbamoyl-9-(phenylmethyl)-2-[[(propen-3-yl)oxy]methyl]carbazol-4-yl]oxyacetic acid; [5-carbamoyl-9-(phenylmethyl)-2-[(propyloxy)methyl]carbazol-4-yl]oxyacetic acid; 9-benzyl-7-methoxy-5-((carboxamidomethyl)oxy)-1,2,3,4-tetrahydrocarbazole-4-carboxamide; 9-benzyl-7-methoxy-5-cyanomethyloxy-carbazole-4-carboxamide; 9-benzyl-7-methoxy-5-((1H-tetrazol-5-yl-methyl)oxy)-carbazole-4-carboxamide; 9-benzyl-7-methoxy-5-((carboxamidomethyl)oxy)-carbazole-4-carboxamide; [9-Benzyl-4-carbamoyl-1,2,3,4-tetrahydrocarbazole-5-yl]oxyacetic acid; {9-[(phenyl)methyl]-5-carbamoyl-2-methyl-carbazol-4-yl}oxyacetic acid; {9-[(3-fluorophenyl)methyl]-5-carbamoyl-2-methylcarbazol-4-yl}oxyacetic acid; {9-[(3-methylphenyl)methyl]-5-carbamoyl-2-methylcarbazol-4-yl}oxyacetic acid; {9-[(phenyl)methyl]-5-carbamoyl-2-(4-trifluoromethylphenyl)-carbazol-4-yl}oxyacetic acid; 9-benzyl-5-(2-methanesulfonamido)ethyloxy-7-methoxy-1,2,3,4-tetrahydrocarbazole-4-carboxamide; 9-benzyl-4-(2-methanesulfonamido)ethyloxy-2-methoxycarbazole-5-carboxamide; 9-benzyl-4-(2-trifluoromethanesulfonamido)ethyloxy-2-methoxycarbazole-5-carboxamide; 9-benzyl-5-methanesulfonamidoylmethyloxy-7-methoxy-1,2,3,4-tetrahydrocarbazole-4-carboxamide; 9-benzyl-4-methanesulfonamidoylmethyloxy-carbazole-5-carboxamide; [5-carbamoyl-2-pentyl-9-(phenylmethyl)carbazol-4-yl]oxyacetic acid; [5-carbamoyl-2-(1-methylethyl)-9-(phenylmethyl)carbazol-4-yl]oxyacetic acid; [5-carbamoyl-9-(phenylmethyl)-2-[(tri(-1-methylethyl)silyl)oxymethyl]carbazol-4-yl]oxyacetic acid; [5-carbamoyl-2-phenyl-9-(phenylmethyl)carbazol-4-yl]oxyacetic acid; [5-carbamoyl-2-(4-chlorophenyl)-9-(phenylmethyl)carbazol-4-yl]oxyacetic acid; [5-carbamoyl-2-(2-furyl)-9-(phenylmethyl)carbazol-4-yl]oxyacetic acid; [5-carbamoyl-9-(phenylmethyl)-2-[(tri(-1-methylethyl)silyl)oxymethyl]carbazol-4-yl]oxyacetic acid; {9-[(3-fluorophenyl)methyl]-5-carbamoylcarbazol-4-yl}oxyacetic acid; {9-[(3-chlorophenyl)methyl]-5-carbamoylcarbazol-4-yl}oxyacetic acid; {9-[(3-phenoxyphenyl)methyl]-5-carbamoylcarbazol-4-yl}oxyacetic acid; {9-[(2-Fluorophenyl)methyl]-5-carbamoylcarbazol-4-yl}oxyacetic acid; {9-[(2-trifluoromethylphenyl)methyl]-5-carbamoylcarbazol-4-yl}oxyacetic acid; {9-[(2-benzylphenyl)methyl]-5-carbamoylcarbazol-4-yl}oxyacetic acid; {9-[(3-trifluoromethylphenyl)methyl]-5-carbamoylcarbazol-4-yl}oxyacetic acid; {9-[(1-naphthyl)methyl]-5-carbamoylcarbazol-4-yl}oxyacetic acid; {9-[(2-cyanophenyl)methyl]-5-carbamoylcarbazol-4-yl}oxyacetic acid; {9-[(3-cyanophenyl)methyl]-5-carbamoylcarbazol-4-yl}oxyacetic acid; {9-[(2-methylphenyl)methyl]-5-carbamoylcarbazol-4-yl}oxyacetic acid; {9-[(3-methylphenyl)methyl]-5-carbamoylcarbazol-4-yl}oxyacetic acid; {9-[(3,5-dimethylphenyl)methyl]-5-carbamoylcarbazol-4-yl}oxyacetic acid; {9-[(3-iodophenyl)methyl]-5-carbamoylcarbazol-4-yl}oxyacetic acid; {9-[(2-Chlorophenyl)methyl]-5-carbamoylcarbazol-4-yl}oxyacetic acid; {9-[(2,3-difluorophenyl)methyl]-5-carbamoylcarbazol-4-yl}oxyacetic acid; {9-[(2,6-difluorophenyl)methyl]-5-carbamoylcarbazol-4-yl}oxyacetic acid; {9-[(2,6-dichlorophenyl)methyl]-5-carbamoylcarbazol-4-yl}oxyacetic acid; {9-[(3-trifluoromethoxyphenyl)methyl]-5-carbamoylcarbazol-4-yl}oxyacetic acid; {9-[(2-biphenyl)methyl]-5-carbamoylcarbazol-4-yl}oxyacetic acid; {9-[(2-Biphenyl)methyl]-5-carbamoylcarbazol-4-yl}oxyacetic acid methyl ester; [9-Benzyl-4-carbamoyl-1,2,3,4-tetrahydrocarbazole-5-yl]oxyacetic acid; {9-[(2-Pyridyl)methyl]-5-carbamoylcarbazol-4-yl}oxyacetic acid; {9-[(3-Pyridyl)methyl]-5-carbamoylcarbazol-4-yl}oxyacetic acid; [9-benzyl-4-carbamoyl-8-methyl-1,2,3,4-tetrahydrocarbazol-5-yl]oxyacetic acid; [9-benzyl-5-carbamoyl-1-methylcarbazol-4-yl]oxyacetic acid; [9-benzyl-4-carbamoyl-8-fluoro-1,2,3,4-tetrahydrocarbazol-5-yl]oxyacetic acid; [9-benzyl-5-carbamoyl-1-fluorocarbazol-4-yl]oxyacetic acid; [9-benzyl-4-carbamoyl-8-chloro-1,2,3,4-tetrahydrocarbazol-5-yl]oxyacetic acid; [9-benzyl-5-carbamoyl-1-chlorocarbazol-4-yl]oxyacetic acid; [9-[(Cyclohexyl)methyl]-5-carbamoylcarbazol-4-yl]oxyacetic acid; [9-[(Cyclopentyl)methyl]-5-carbamoylcarbazol-4-yl]oxyacetic acid; [5-carbamoyl-9-(phenylmethyl)-2-(2-thienyl)carbazol-4-yl]oxyacetic acid; [5-carbamoyl-9-(phenylmethyl)-2-[[(propen-3-yl)oxy]methyl]carbazol-4-yl]oxyacetic acid; [5-carbamoyl-9-(phenylmethyl)-2-[(propyloxy)methyl]carbazol-4-yl]oxyacetic acid; 9-benzyl-7-methoxy-5-((carboxamidomethyl)oxy)-1,2,3,4-tetrahydrocarbazole-4-carboxamide; 9-benzyl-7-methoxy-5-cyanomethyloxy-carbazole-4-carboxamide; 9-benzyl-7-methoxy-5-((1H-tetrazol-5-yl-methyl)oxy)-carbazole-4-carboxamide; 9-benzyl-7-methoxy-5-((carboxamidomethyl)oxy)-carbazole-4-carboxamide; [9-Benzyl-4-carbamoyl-1,2,3,4-tetrahydrocarbazole-5-yl]oxyacetic acid; (R,S)-(9-benzyl-4-carbamoyl-1-oxo-3-thia-1,2,3,4-tetrahydrocarbazol-5-yl)oxyacetic acid; (R,S)-(9-benzyl-4-carbamoyl-3-thia-1,2,3,4-tetrahydrocarbazol-5-yl)oxyacetic acid; 2-(4-oxo-5-carboxamido-9-benzyl-9H-pyrido[3,4-b]indolyl)acetic acid chloride; [N-benzyl-1-carbamoyl-1-aza-1,2,3,4-tetrahydrocarbazol-8-yl]oxyacetic acid; 4-methoxy-6-methoxycarbonyl-10-phenylmethyl-6,7,8,9-tetrahydropyrido[1,2-a]indole; (4-carboxamido-9-phenylmethyl-4,5-dihydrothiopyrano[3,4-b]indol-5-yl)oxyacetic acid; 3,4-dihydro-4-carboxamidol-5-methoxy-9-phenylmethylpyrano[3,4-b]indole; 2-[(2,9 bis-benzyl-4-carbamoyl-1,2,3,4-tetrahydro-betacarbolin-5-yl)oxy]acetic acid; 2-[4-oxo-5-carboxamido-9-(2-methylbenzyl)-9H-pyrido[3,4-b]indolyl]acetic acid; 2-[4-oxo-5-carboxamido-9-(3-methylbenzyl)-9H-pyrido[3,4-b]indolyl]acetic acid; 2-[4-oxo-5-carboxamido-9-(4-methylbenzyl)-9H-pyrido[3,4-b]indolyl]acetic acid; 2-[4-oxo-5-carboxamido-9-(4-tert-butylbenzyl)-9H-pyrido[3,4-b]indolyl]acetic acid; 2-[4-oxo-5-carboxamido-9-pentafluorobenzyl-9H-pyrido[3,4-b]indolyl]acetic acid; 2-[4-oxo-5-carboxamido-9-(2-fluorobenzyl)-9H-pyrido[3,4-b]indolyl]acetic acid; 2-[4-oxo-5-carboxamido-9-(3-fluorobenzyl)-9H-pyrido[3,4-b]indolyl]acetic acid; 2-[4-oxo-5-carboxamido-9-(4-fluorobenzyl)-9H-pyrido[3,4-b]indolyl]acetic acid; 2-[4-oxo-5-carboxamido-9-(2,6-difluorobenzyl)-9H-pyrido[3,4-b]indolyl]acetic acid; 2-[4-oxo-5-carboxamido-9-(3,4-difluorobenzyl)-9H-pyrido[3,4-b]indolyl]acetic acid; 2-[4-oxo-5-carboxamido-9-(2,5-difluorobenzyl)-9H-pyrido[3,4-b]indolyl]acetic acid; 2-[4-oxo-5-carboxamido-9-(3,5-difluorobenzyl)-9H-pyrido[3,4-b]indolyl]acetic acid; 2-[4-oxo-5-carboxamido-9-(2,4-difluorobenzyl)-9H-pyrido[3,4-b]indolyl]acetic acid; 2-[4-oxo-5-carboxamido-9-(2,3-difluorobenzyl)-9H-pyrido[3,4-b]indolyl]acetic acid; 2-[4-oxo-5-carboxamido-9-[2-(trifluoromethyl)benzyl]-9H-pyrido[3,4-b]indolyl]acetic acid; 2-[4-oxo-5-carboxamido-9-[2-(trifluoromethyl)benzyl]-9H-pyrido[3,4-b]indolyl]acetic acid; 2-[4-oxo-5-carboxamido-9-[3-(trifluoromethyl)benzyl]-9H-pyrido[3,4-b]indolyl]acetic acid; 2-[4-oxo-5-carboxamido-9-[4-(trifluoromethyl)benzyl]-9H-pyrido[3,4-b]indolyl]acetic acid; 2-[4-oxo-5-carboxamido-9-[3,5-bis(trifluoromethyl)benzyl]-9H-pyrido[3,4-b]indolyl]acetic acid; 2-[4-oxo-5-carboxamido-9-[2,4-bis(trifluoromethyl)benzyl]-9H-pyrido[3,4-b]indolyl]acetic acid; 2-[4-oxo-5-carboxamido-9-(a-methylnaphthyl)-9H-pyrido[3,4-b]indolyl]acetic acid; 2-[4-oxo-5-carboxamido-9-(b-methylnaphthyl)-9H-pyrido[3,4-b]indolyl]acetic acid; 2-[4-oxo-5-carboxamido-9-(3,5-dimethylbenzyl)-9H-pyrido[3,4-b]indolyl]acetic acid; 2-[4-oxo-5-carboxamido-9-(2,4-dimethylbenzyl)-9H-pyrido[3,4-b]indolyl]acetic acid; 2-[4-oxo-5-carboxamido-9-(2-phenylbenzyl)-9H-pyrido[3,4-b]indolyl]acetic acid; 2-[4-oxo-5-carboxamido-9-(3-phenylbenzyl)-9H-pyrido[3,4-b]indolyl]acetic acid; 2-[4-oxo-5-carboxamido-9-(4-phenylbenzyl)-9H-pyrido[3,4-b]indolyl]acetic acid; 2-[4-oxo-5-carboxamido-9-(1-fluorenylmethy)-9H-pyrido[3,4-b]indolyl]acetic acid; 2-[4-oxo-5-carboxamido-9-(2-fluoro-3-methylbenzyl)-9H-pyrido[3,4-b]indolyl]acetic acid; 2-[4-oxo-5-carboxamido-9-(3-benzoylbenzyl)-9H-pyrido[3,4-b]indolyl]acetic acid; 2-[4-oxo-5-carboxamido-9-(2-phenoxybenzyl)-9H-pyrido[3,4-b]indolyl]acetic acid; 2-[4-oxo-5-carboxamido-9-(3-phenoxybenzyl)-9H-pyrido[3,4-b]indolyl]acetic acid; 2-[4-oxo-5-carboxamido-9-(4-phenoxybenzyl)-9H-pyrido[3,4-b]indolyl]acetic acid; 2-[4-oxo-5-carboxamido-9-[3-[2-(fluorophenoxy)benzyl]]-9H-pyrido[3,4-b]indolyl]acetic acid; 2-[4-oxo-5-carboxamido-9-[3-[4-(fluorophenoxy)benzyl]]-9H-pyrido[3,4-b]indolyl]acetic acid; 2-[4-oxo-5-carboxamido-9-[2-fluoro-3-(trifluoromethyl)benzyl]-9H-pyrido[3,4-b]indolyl]acetic acid; 2-[4-oxo-5-carboxamido-9-[2-fluoro-4-(trifluoromethyl)benzyl]-9H-pyrido[3,4-b]indolyl]acetic acid; 2-[4-oxo-5-carboxamido-9-[2-fluoro-5-(trifluoromethyl)benzyl]-9H-pyrido[3,4-b]indolyl]acetic acid; 2-[4-oxo-5-carboxamido-9-[3-fluoro-5-(trifluoromethyl)benzyl]-9H-pyrido[3,4-b]indolyl]acetic acid; 2-[4-oxo-5-carboxamido-9-[4-fluoro-2-(trifluoromethyl)benzyl]-9H-pyrido[3,4-b]indolyl]acetic acid; 2-[4-oxo-5-carboxamido-9-[4-fluoro-3-(trifluoromethyl)benzyl]-9H-pyrido[3,4-b]indolyl]acetic acid; 2-[4-oxo-5-carboxamido-9-[2-fluoro-6-(trifluoromethyl)benzyl]-9H-pyrido[3,4-b]indolyl]acetic acid; 2-[4-oxo-5-carboxamido-9-(2,3,6-trifluorobenzyl)-9H-pyrido[3,4-b]indolyl]acetic acid; 2-[4-oxo-5-carboxamido-9-(2,3,5-trifluorobenzyl)-9H-pyrido[3,4-b]indolyl]acetic acid; 2-[4-oxo-5-carboxamido-9-(2,4,5-trifluorobenzyl)-9H-pyrido[3,4-b]indolyl]acetic acid; 2-[4-oxo-5-carboxamido-9-(2,4,6-trifluorobenzyl)-9H-pyrido[3,4-b]indolyl]acetic acid; 2-[4-oxo-5-carboxamido-9-(2,3,4-trifluorobenzyl)-9H-pyrido[3,4-b]indolyl]acetic acid; 2-[4-oxo-5-carboxamido-9-(3,4,5-trifluorobenzyl)-9H-pyrido[3,4-b]indolyl]acetic acid; 2-[4-oxo-5-carboxamido-9-[3-(trifluoromethoxyl)benzyl]-9H-pyrido[3,4-b]indolyl]acetic acid; 2-[4-oxo-5-carboxamido-9-[4-(trifluoromethoxyl)benzyl]-9H-pyrido[3,4-b]indolyl]acetic acid; 2-[4-oxo-5-carboxamido-9-[4-methoxy(tetrafluoro)benzyl]-9H-pyrido[3,4-b]indolyl]acetic acid; 2-[4-oxo-5-carboxamido-9-(2-methoxybenzyl)-9H-pyrido[3,4-b]indolyl]acetic acid; 2-[4-oxo-5-carboxamido-9-(3-methoxybenzyl)-9H-pyrido[3,4-b]indolyl]acetic acid; 2-[4-oxo-5-carboxamido-9-(4-methoxybenzyl)-9H-pyrido[3,4-b]indolyl]acetic acid; 2-[4-oxo-5-carboxamido-9-(4-ethylbenzyl)-9H-pyrido[3,4-b]indolyl]acetic acid; 2-[4-oxo-5-carboxamido-9-(4-isopropylbenzyl)-9H-pyrido[3,4-b]indolyl]acetic acid; 2-[4-oxo-5-carboxamido-9-(3,4,5-trimethoxybenzyl)-9H-pyrido[3,4-b]indolyl]acetic acid; 2-[4-oxo-5-carboxamido-9-(3,4-methylenedioxybenzyl)-9H-pyrido[3,4-b]indolyl]acetic acid; 2-[4-oxo-5-carboxamido-9-(4-methoxy-3-methylbenzyl)-9H-pyrido[3,4-b]indolyl]acetic acid; 2-[4-oxo-5-carboxamido-9-(3,5-dimethoxybenzyl)-9H-pyrido[3,4-b]indolyl]acetic acid; 2-[4-oxo-5-carboxamido-9-(2,5-dimethoxybenzyl)-9H-pyrido[3,4-b]indolyl]acetic acid; 2-[4-oxo-5-carboxamido-9-(4-ethoxybenzyl)-9H-pyrido[3,4-b]indolyl]acetic acid; 2-[4-oxo-5-carboxamido-9-(cyclohexylmethyl)-9H-pyrido[3,4-b]indolyl]acetic acid; 2-[4-oxo-5-carboxamido-9-(cyclopentylmethyl)-9H-pyrido[3,4-b]indolyl]acetic acid; 2-[4-oxo-5-carboxamido-9-ethyl-9H-pyrido[3,4-b]indolyl]acetic acid; 2-[4-oxo-5-carboxamido-9-(1-propyl)-9H-pyrido[3,4-b]indolyl]acetic acid; 2-[4-oxo-5-carboxamido-9-(2-propyl)-9H-pyrido[3,4-b]indolyl]acetic acid; 2-[4-oxo-5-carboxamido-9-(1-butyl)-9H-pyrido[3,4-b]indolyl]acetic acid; 2-[4-oxo-5-carboxamido-9-(2-butyl)-9H-pyrido[3,4-b]indolyl]acetic acid; 2-[4-oxo-5-carboxamido-9-isobutyl-9H-pyrido[3,4-b]indolyl]acetic acid; 2-[4-oxo-5-carboxamido-9-[2-(1-phenylethyl)]-9H-pyrido[3,4-b]indolyl]acetic acid; 2-[4-oxo-5-carboxamido-9-[3-(1-phenylpropyl)]-9H-pyrido[3,4-b]indolyl]acetic acid; 2-[4-oxo-5-carboxamido-9-[4-(1-phenylbutyl)]-9H-pyrido[3,4-b]indolyl]acetic acid; 2-[4-oxo-5-carboxamido-9-(1-pentyl)-9H-pyrido[3,4-b]indolyl]acetic acid; 2-[4-oxo-5-carboxamido-9-(1-hexyl)-9H-pyrido[3,4-b]indolyl]acetic acid; 4-[(9-benzyl-4-carbamoyl-1,2,3,4-tetrahydrocarbazol-6-yl)oxy]butyric acid; 3-[(9-benzyl-4-carbamoyl-1,2,3,4-tetrahydrocarbazol-6-yl)oxy]propylphosphonic acid; 2-[(9-benzyl-4-carbamoyl-1,2,3,4-tetrahydrocarbazol-6-yl)oxy]methylbenzoic acid; 3-[(9-benzyl-4-carbamoyl-7-n-octyl-1,2,3,4-tetrahydrocarbazol-6-yl)oxy]propylphosphonic acid; 4-[(9-benzyl-4-carbamoyl-7-ethyl-1,2,3,4-tetrahydrocarbazol-6-yl)oxy]butyric acid; 3-[(9-benzyl-4-carbamoyl-7-ethyl-1,2,3,4-tetrahydrocarbazol-6-yl)oxy]propylphosphonic acid; 3-[(9-benzyl-4-carbamoyl-7-ethyl-1,2,3,4-tetrahydrocarbazol-6-yl)oxy]propylphosphonic acid; (S)-(+)-4-[(9-benzyl-4-carbamoyl-7-ethyl-1,2,3,4-tetrahydrocarbazol-6-yl)oxy]butyric acid; 4-[9-benzyl-4-carbamoyl-6-(2-cyanoethyl)-1,2,3,4-tetrahydrocarbazol-6-yl]oxybutyric acid; 4-[9-benzyl-4-carboxamido-7-(2-phenylethyl)-1,2,3,4-tetrahydrocarbazol-6-yl]oxybutyric acid; 4-[9-benzyl-4-carboxamidocarbazol-6-yl]oxybutyric acid; methyl 2-[(9-benzyl-4-carbamoyl-1,2,3,4-tetrahydrocarbazol-6-yl)oxy]methylbenzoate; 4-[9-benzyl-4-carbamoyl-7-(2-cyanoethyl)-1,2,3,4-tetrahydrocarbazol-6-yl]oxybutyric acid; 9-benzyl-7-methoxy-5-cyanomethyloxy-1,2,3,4-tetrahydrocarbazole-4-carboxamide; [9-benzyl-4-carbamoyl-8-methyl-carbazole-5-yl]oxyacetic acid; and [9-benzyl-4-carbamoyl-carbazole-5-yl]oxyacetic acid, or pharmaceutically acceptable salts, solvates, prodrug derivatives, racemates, tautomers, or optical isomers thereof.

In certain embodiments, the conjugate compounds provided herein may comprise A-001 linked to a compound used in the treatment of CVD by an anhydride or acetal/ketal linkage. In other embodiments, different chemical linkages known in the art may be used.

In certain embodiments, the conjugate compounds provided herein may be synthesized as anhydrides (see, e.g., Roulleau 1983). For example, an A-001/niacin conjugate may be generated as an anhydride in the following manner:

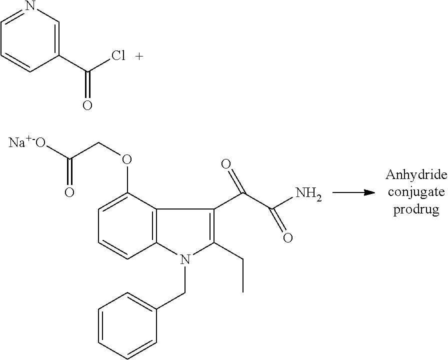

In other embodiments, the conjugate compounds provided herein may be synthesized as acetals or ketals (see, e.g., Frampton 1992; Blouin 1993; Imada 1995; Perry 1996; Fujimoto 2001). For example, an A-001/niacin conjugate may be generated as an acetal/ketal in the following manner:

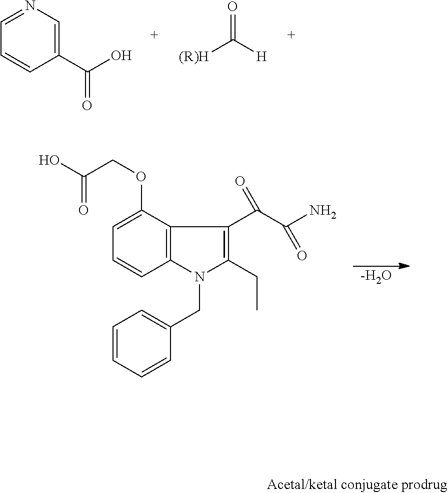

In certain embodiments, an A-001/niacin conjugate compound as provided herein is a prodrug having the chemical formula C₂₇H₂₃N₃O₆ and the chemical structure:

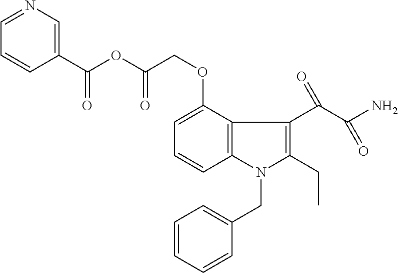

In other embodiments, an A-001/niacin conjugate compound as provided herein is a prodrug having the chemical formula C₂₈H₂₅N₃O₇ and the chemical structure:

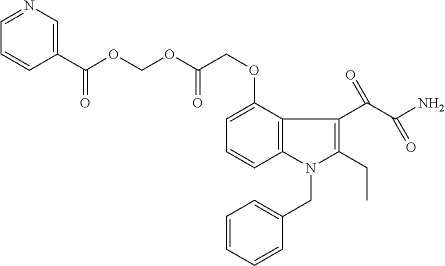

In still other embodiments, an A-001/niacin conjugate compound as provided herein is a prodrug having the chemical formula C₂₉H₂₇N₃O₇ and the chemical structure:

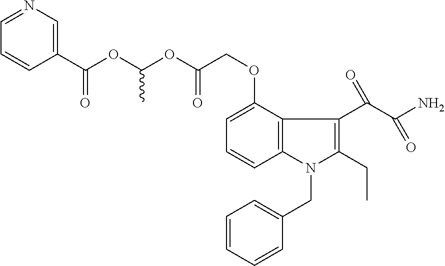

Provided herein in certain embodiments are methods of altering lipid levels in a subject comprising administering a conjugate compound as provided herein. In certain of these embodiments, methods are provided for decreasing TG levels and/or increasing HDL levels in a subject in need thereof by administering one or more of the A-001/niacin conjugate prodrugs disclosed herein. In other embodiments, methods are provided for decreasing total cholesterol, non-HDL cholesterol, LDL, LDL particle, small LDL particle, oxidized LDL, and/or ApoB levels in a subject in need thereof by administering one or more of the A-001/statin conjugate prodrugs disclosed herein.

Provided herein in certain embodiments are methods of treating a CVD associated with elevated TG levels and/or low HDL levels in a subject comprising administering a conjugate compound as provided herein. In certain of these embodiments, methods are provided for treating a CVD associated with elevated TG levels and/or low HDL levels by administering one or more of the A-001/niacin conjugate prodrugs disclosed herein.

In certain embodiments, formulations are provided that comprise one or more of the conjugate compounds as disclosed herein in combination with one or more inactive ingredients. In certain of these embodiments, the formulations may be in the form of an oral dosage unit such as a tablet, capsule, or pill. In certain embodiments, formulations may comprise one or more additional active ingredients, such as other compounds used in the treatment of CVD.

Further provided herein are kits comprising one or more of the conjugate compounds as disclosed herein, as well as the use of such kits in altering lipid levels or treating CVD in a subject. In certain embodiments, these kits further comprise instructions for usage, such as dosage or administration instructions. In certain embodiments, these kits may comprise one or more additional active ingredients, such as other compounds used in the treatment of CVD.

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Example 1

### Synthesis

A-001 or A-002 will be conjugated to niacin or other niacin drugs to generate one or more A-001/niacin conjugate compounds using standard chemical synthesis methods. Initial conjugate compounds will be synthesized as anhydrides or acetal/ketals as follows:

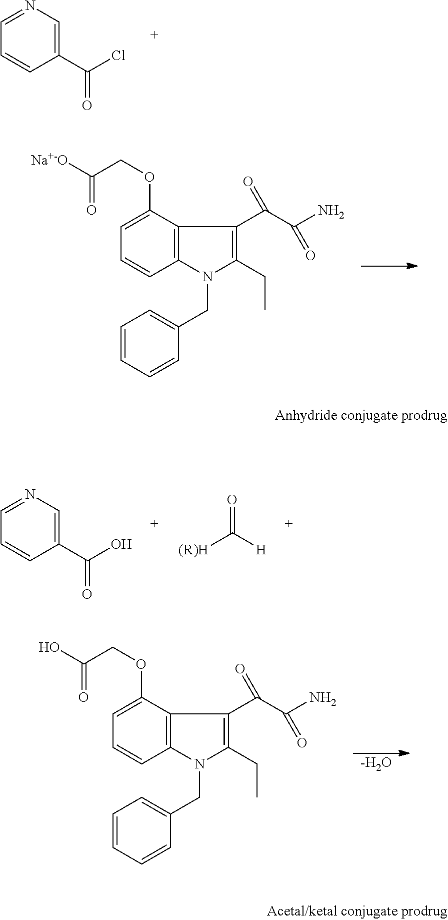

### Example 2

### In Vitro Characterization

One or more of the conjugate compounds generated in Example 1 will undergo initial characterization using an array of standard analytical techniques. These may include one or more of the following: scanning calorimetry, X-ray diffraction, multi-nuclear NMR, chromatographic techniques, IR and Raman spectroscopy, mass spectroscopy, or other standard analytical techniques known in the art.

In addition, conjugate compounds will be tested for chemical and physical stability under commonly used stressing conditions, including temperature, humidity, light, oxygen, other oxidizing conditions, acid and base treatment, and across a range of pH's. Solubility will also be assessed using standard methods. CACO modeling may be performed to assess potential cellular membrane flux and bioavailability.

### Example 3

### Formulations

One or more of the conjugate compounds generated in Example 1 will be formulated into fixed dose formulations using methods known in the art. Previous studies have established the feasibility of formulating A-002 into a tablet comprising the inactive ingredients lactose, hydroxypropyl cellulose, croscarmellose sodium, polysorbate 80, microcrystalline cellulose and magnesium state, while niacin is commonly formulated in an extended release dosage using the inactive ingredients hydroxypropyl methylcellulose, povidone, steric acid and polyethylene glycol. Conjugate compound formulations will utilize various combinations of one or more of these inactive ingredients. Additional inactive components such as calcium carbonate may be added to conjugate compound formulations as well.

Conjugate compounds may be formulated into a tablet, capsule, implantable wafer or disc, or other forms known in the art. Formulations may be designed for immediate release of the conjugate compound, or they may be designed for extended or delayed release using commonly known excipients and technology. Extended release formulations may comprise a plurality of particles or beads with a distintegratable coating, wherein the conjugate compounds are incorporated into or distributed along the surface of each particle or bead. Formulations may contain particles or beads with a variety of coating thicknesses, such that the conjugate compounds are released at different times following administration. Such formulations may result in substantially constant blood levels of conjugate compounds over an extended period. Alternatively, these formulations may result in a pulsed plasma profile, wherein the conjugate compounds are released in cycles. Formulations should be designed in such a way as to deliver an effective dosage of conjugate compounds.

In addition to conjugate compounds, formulations may incorporate one or more additional active ingredients such as one or more statins, including atorvastatin, cerivastatin, fluvastatin lovastatin, mevastatin, pitavastatin, pravastatin, rosuvastatin, or simvastatin. Commonly available excipients that are routinely used for the formulation of statins into solid oral dosage forms may be incorporated into these formulations.

### Example 4

### In Vivo Characterization

Conjugate compounds and conjugate compound formulations will be tested in one or more animal models for in vivo characteristics such as toxicity and bioavailability. Once this characterization is complete, the compounds and formulations will be tested for efficacy (e.g., ability to alter serum lipid levels) in one or more animal or human models.

As stated above, the foregoing is merely intended to illustrate various embodiments of the present invention. The specific modifications discussed above are not to be construed as limitations on the scope of the invention. It will be apparent to one skilled in the art that various equivalents, changes, and modifications may be made without departing from the scope of the invention, and it is understood that such equivalent embodiments are to be included herein. All references cited herein are incorporated by reference as if fully set forth herein.

### REFERENCES

1. Blouin Clin Pharmacokinet 25:172 (1993)
2. Boekholdt Arterioscler Thromb Vasc Biol 25:839 (2005)
3. Bostrom Arterioscler Thromb Vasc Biol 27:600 (2007)
4. Camejo Atherosclerosis 139:205 (1998)
5. Chait J Lipid Res 46:389 (2005)
6. Elinder Arterioscler Thromb Vasc Biol 17:2257 (1997)
7. Frampton Drugs 44:889 (1992)
8. Fujimoto Int J Antimicrob Agents 18:489 (2001)
9. Hakala Arterioscler Thromb Vasc Biol 21:1053 (2001)
10. Hartford J Cardiol 108:55 (2006)
11. Hurt-Camejo Arterioscler Thromb Vasc Biol 17:300 (1997)
12. Imada Int J Antimicrob Agents 5:85 (1995)
13. Ivandic Arterioscler Thromb Vasc Biol 19:1284 (1999)
14. Kimura-Matsumoto Atherosclerosis 196:81 (2007)
15. Kugiyama Circulation 100:1280 (1999)
16. Liu Eur Heart J 24:1824 (2003)
17. Mallat J Am Coll Cardiol 46:1249 (2005)
18. Mallat Arterioscler Thromb Vasc Biol 27:1177 (2007)
19. Menschikowski Atherosclerosis 118:173 (1995)
20. Nijmeijer Cardiovasc Res 53:138 (2002)
21. Perry Drugs 52:125 (1996)
22. Pruzanski J Lipid Res 39:2150 (1998)
23. Rosengren Arterioscler Thromb Vasc Biol 26:1579 (2006)
24. Roulleau Tetrahedron Lett 24:4195 (1983)
25. Sartipy J Biol Chem 274:25913 (1999)
26. Serruys Circulation 118:1172 (2008)
27. Tietge J Biol Chem 275:10077 (2000)

What is claimed is:

1. Composition of matter comprising A-001 or a prodrug, salt, solvate, or stereoisomer thereof linked to a compound used in the treatment of CVD via a standard chemical linkage to form a conjugate prodrug.

2. The composition of matter of claim 1, wherein the compound used in the treatment of CVD is a niacin drug selected from the group consisting of niacin, nicotinic acid, and acipimox.

3. The composition of matter of claim 1, wherein the conjugate prodrug is an anhydride.

4. The composition of matter of claim 1, wherein the conjugate prodrug is an acetal or ketal.

5. The composition of matter of claim 2, wherein the conjugate prodrug has the structure:

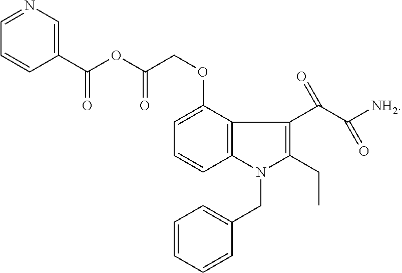

6. The composition of matter of claim 2, wherein the conjugate prodrug has the structure:

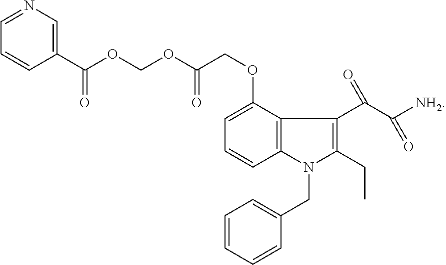

7. The composition of matter of claim 2, wherein the conjugate prodrug has the structure:

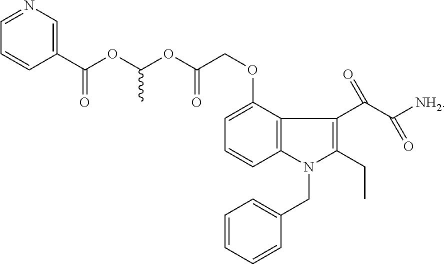

8. A method of altering lipid levels in a subject in need thereof comprising administering the conjugate prodrug of claim 1.

9. The method of claim 8, wherein administration of said conjugate prodrug results in one or more of a reduction in total cholesterol, LDL, LDL particle, small LDL particle, or triglyceride levels or an increase in HDL levels.
